(19) United States
(12) Patent Application Publication
(10) Pub. No.: US 2026/0188446 A1
(43) Pub. Date: Jul. 2, 2026

(54) COMPUTER-IMPLEMENTED METHOD FOR STORING MEDICAL DATA

(71) Applicant: Siemens Healthineers AG, Forchheim (DE)

(72) Inventor: Wolfgang Bielmeier, Erlangen (DE)

(21) Appl. No.: 19/434,527

(22) Filed: Dec. 29, 2025

(30) Foreign Application Priority Data
Dec. 30, 2024 (DE) 10 2024 212 332.3

(51) Int. Cl. G16H 10/60; G06F 16/901

(52) U.S. Cl. CPC G16H 10/60; G06F 16/9024

(57) ABSTRACT

A computer-implemented method for storing medical data. One of the methods comprises: continuously receiving sequentially created medical data packets, each of which comprises a start time point, an end time point and a data packet value which defines a total of medical measurement values acquired between the start time point and the end time point; for each received medical data packet, determining a total value by adding the data packet value according to the respective medical data packet with the data packet values of at least a part of the received medical data packets created temporally before the respective medical data packet; and storing medical data by storing the respective medical data packet together with the respectively determined total value in a linked list and/or in a data field.

### CROSS REFERENCE TO RELATED APPLICATIONS

This application claims the benefit of DE 10 2024 212 332.3 filed on Dec. 30, 2024, which is hereby incorporated by reference in its entirety.

### FIELD

Embodiments relate to a computer-implemented method for storing medical data.

### BACKGROUND

In the context of a medical investigation and/or treatment, at least one medical measurement value may be acquired. The medical measurement value is, for example, during an execution of a magnetic resonance tomography (MRI), a specific absorption rate (SAR), being the energy absorbed per unit time and mass in a body of a patient from electromagnetic radiation that is emitted by an MRI device. The medical measurement value is continuously monitored during the medical investigation and/or treatment in order, for example, to recognize when the absorbed energy from electromagnetic radiation reaches or exceeds a specified limit value. For this purpose, for example, continuously acquired medical measurement values are stored and evaluated one after the other in a list. Given numerous measurement values and a time-span of, for example, several minutes that the medical investigation and/or treatment takes, this may lead to an extensive list so that a targeted retrieval of individual entries in the list may be time-intensive.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art. Independent of the grammatical term usage, individuals with male, female or other gender identities are included within the term.

Embodiments provide a solution by which stored medical data may be made quickly accessible for an evaluation.

A first aspect relates to a computer-implemented method for storing medical data. The computer-implemented method is carried out, for example, by a computer or a computing facility, for example, a data processing system.

Embodiments provide where medical data such as, for example, a specific absorption rate (SAR), may be stored, for example, in doubly linked lists. Each element of the doubly linked list includes a start time point, an end time point and a data packet value that defines a sum of medical measurement values acquired between the start time point and the end time point, for example, acquired SAR values. Furthermore, a first pointer and a second pointer are provided that point to a directly following element or a directly preceding element of the doubly linked list. With the aid of the pointers, the individual elements of the doubly linked list may be recalled one after the other, that is, the elements of the doubly linked list may only be reached one after the other without the possibility of jumping over at least a part of the elements. In order to establish a total value from a plurality of individual data packet values, the elements must be retrieved and read out one after the other. An evaluation of the medical data that is described by the doubly linked list is therefore time-consuming and possibly computation-intensive.

The method includes continuous receiving of successively generated medical data packets. Each medical data packet includes a start time point, an end time point and a data packet value. The data packet value defines a sum of medical measurement values acquired between the start time point and the end time point. The data packet value may be understood to be an integral over the medical measurement values between the start point and the end point. If, between the start time point and the end time point, a specified number of medical measurement values was acquired, they may be summed in order to determine the data packet value so that, in this example, no function is determined that defines a course of the medical measurement values between the start time point and the end time point. The start time point and the end time point are given, for example, as absolute time values, for example, as times of day. If, for example, the acquisition of medical measurement values is begun at 10:05 hours, the start time point of the first medical data packet may be 10:05 and the end time point of the first medical data packet may be 5 seconds after 10:05 if, for example, a medical data packet is received in each time window of 5 seconds length. In a preferred example, a plurality of medical data packets are generated per second and are continuously received one after the other. The start time point and the end time point may alternatively be understood as relative time values that start at 0 for the first medical data packet. The start time point of a directly following medical data packet is, at a preferred time point, the end time point of the directly preceding medical data packet, so that no time gap exists between the individual medical data packets.

For each medical data packet, a determination of a total value takes place. The total value is determined individually for all the medical data packets received. Each total value may be calculated by adding the respective data packet value according to the respective medical data packet with the data packet values of at least a part of the received medical data packets created temporally before the respective medical data packet. Thus, for a currently observed medical data packet, the data packet value associated with this medical data packet is summed with the data packet values of preceding data packets, for example, of all the preceding data packets from the first medical data packet. Thus, all the previously determined medical data packets and the currently observed medical data packet may be taken into consideration when the total value is calculated. The total value is thus the total of a plurality of individual data packet values. The total value may alternatively be calculated by adding the data packet value of the currently observed data packet to the total value that was determined for the directly preceding data packet.

The computer-implemented method includes storing medical data by storing the respective medical data packet together with the respectively determined total value in a linked list and/or in a data field. Thus, for each medical data packet, the start time point, the end time point and the data packet value are stored and, in addition, the total value for this medical data packet itself. For each medical data packet, the linked list and/or the data field therefore has, for example, at least four entries, that is, the start time point, the end time point, the data packet value and the total value.

The linked list may be understood to be a dynamic data structure in which individual data elements are stored in order. The individual data elements are referred to below as elements. When a linked list is created, it is not necessary for a maximum number of the elements and therefore a maximum number of received medical data packets to be specified, but rather the maximum number may be selected as desired during the execution of the computer-implemented method.

The data field may alternatively be designated a field or array. The data field is a data structure on use of which similarly structured packets of data may be processed. Each packet includes, for example, at least the start time point, the end time point and the data packet value of one of the medical data packets and also the total value determined for this data packet. With the aid of indices, packets of the data field may be accessed.

By way of the determination of the total value for each medical data packet and its storage in the linked list and/or in the data field, for each data packet, the sum of the individual data packet values is stored so that, for example, on requesting or approaching an x-th entry in the linked list and/or in the data field, the current total value may be read out without this still needing to be calculated. This leads to time savings in the evaluation of the stored medical data. Stored medical data is therefore made quickly available for an evaluation.

One embodiment provides that the medical measurement values give a specific absorption rate (SAR). The specific absorption rate defines a level of energy absorbed per unit time and mass in a body of a patient by way of electromagnetic radiation during a medical investigation and/or treatment. The medical investigation and/or treatment includes, for example, a magnetic resonance tomography (MRI) with an MRI device during which magnetic images and/or radio wave sectional images of at least a part of the body of the patient are created. For example, in the case of ultra-high field MRI or high field MRI, a specified limit value for the specific absorption rate during the medical investigation and/or treatment may be achieved. The achievement and possibly an exceeding of the predetermined limit value is therefore monitored, for example, during the medical investigation and/or treatment. The specific absorption rate is therefore particularly meaningful as a medical measurement value, and it may nevertheless be reliably achieved with the computer-implemented method.

A further embodiment includes that at least one time interval is specified. On determination of the total value for the respective medical data packet, only the received medical data packets are taken into account, the start time point and/or end time point of which is in the same specified time interval as the respective medical data packet. Thus, larger time intervals may be selected that are greater than the spacing between the start time point and the end time point of an individual medical data packet and thus the total value for the whole time interval is determined by simple means. The total value for the time interval may, for example, additionally be stored and/or evaluated in a targeted manner. Herein, the total value determined for the at least one time interval may, for example, be compared with a limit value specified for this time interval, wherein after the specified limit value is reached, for example, a notification message is output and/or the medical investigation and/or treatment is automatically ended or paused. Thus, on the basis of the time interval for a further evaluation, suitable total values may be determined and thereby still greater time savings may be achieved.

In a further embodiment, the at least one specified time interval is 10 seconds long. Alternatively or additionally, the at least one specified time interval is 6 minutes long. Alternative or additional time intervals are possible, for example, between more than 10 seconds and less than 1 minute, between 1 minute and less than 6 minutes, for example, between 3 minutes and 5 minutes or between more than 6 minutes and 10 minutes. In one example, a time interval of less than 10 seconds or more than 10 minutes may be specified. It may thus be provided that at least every 10 seconds and/or every 6 minutes, the total value is determined and, for example, stored in order to be able to organize the monitoring of the medical measurement values into useful time interval packets.

In another embodiment, it is provided that the medical data is stored in the linked list. It is also provided that each medical data packet with the determined total value forms an element of the linked list. At least one first pointer to a following element and at least one second pointer to a preceding element is associated with the respective element. An entry in the linked list, that is, the respective element, may thus be enhanced with at least two different pointers that here are designated the first and second pointer. A pointer is a capability for linking individual elements of the linked list to one another. For example, in a doubly linked list, each element has both a pointer to the following element and also a pointer to the preceding element. The first pointer may therefore alternatively be designated a following item pointer and the second pointer may be designated a preceding item pointer. The advantage of the use of pointers is that individual elements of the linked list may be reached relatively easily, since the pointers to a storage location of the elements to which the pointer points may be followed. Therefore, in the linked list, considered in terms of time, starting from one element, it is possible to move forward or backward to reach other elements and read them. This is a particularly useful possibility for being able to access the different sequentially created medical data packets and/or the total values determined for them, even if the individual elements are not stored in contiguous storage locations.

Furthermore, one embodiment provides that the following element includes the medical data packet that was acquired directly after the respective medical data packet. The preceding element includes the medical data packet that was acquired directly before the respective medical data packet. Directly, in this context, means immediately or without any intermediate element. Thus, two pointers may be provided that point to the directly following and/or to the directly preceding element and thus to the data packet stored in this element with the total value and are therefore linked to it. This has the result that the linked list may be traversed iteratively from front to back or from back to front, starting from any desired element in order, for example, to reach a particular total value and/or to determine a chronological sequence of the total values.

In another embodiment, it may be provided that the following element includes the medical data packet that was acquired a specified number of acquisition steps after the respective medical data packet, and/or the preceding element includes the medical data packet that was acquired the specified number of acquisition steps before the respective medical data packet. Thus, pointers may be provided that point to elements of the linked list that do not directly adjoin the currently observed element, but are removed from it by the specified number of acquisition steps. The number of acquisition steps may be any desired number. In one example with a total of several hundred medical data packets and therefore elements, the number of the acquisition steps may be, for example, 35. This means that, starting from an observed element, it is possible to change to an element 35 elements later or earlier. This has the result that, for example, for a total of 500 individual elements and the number of acquisition steps at 35, a middle element, that is, the element number 250 of 500, may be reached by way of 7 steps according to the second pointer, with which 34 elements are jumped over in each case, and 5 individual steps to directly following elements. In total, in this example, 12 steps are needed and not 250 individual steps to directly following elements in each case. This leads to a reduction in the steps needed by more than 90 percent, so that a significant time saving is to be expected. Another number of acquisition steps than 35 is possible, for example, a larger or smaller number. Therefore, by the pointer described, further time savings are possible.

A further aspect includes a computer-implemented method for storing medical data. The computer-implemented method includes a continuous receiving of successively created medical data packets, each of which includes a start time point, an end time point and a data packet value that defines a sum of medical measurement values acquired between the start time point and the end time point. The computer-implemented method includes storing medical data by storing each medical data packet in a linked list. Each medical data packet forms an element of the linked list. The determination of the total value and its storage together with the respective medical data packet is therefore not an obligatory method step of this computer-implemented method.

For each element of the linked list, an allocation of at least one first pointer to a following element and at least one second pointer to a preceding element takes place. The following element includes the medical data packet that was created a specified number of acquisition steps after the respective medical data packet. The preceding element includes the medical data packet that was created a specified number of acquisition steps before the respective medical data packet. Therefore, the large steps are explicitly provided between the individual data packets that may be selected by a pointer. This has the advantage that a time saving is achieved since not every individual directly successive element is mutually linked by the pointer, but rather, by way of the pointer, it is possible to change by larger steps between the medical data packets stored in the linked list. The stored medical data is therefore made quickly accessible for an evaluation.

With both the described computer-implemented methods, it is therefore possible to reduce a time expenditure for the navigation within the stored medical data and/or for the evaluation of the medical data.

One embodiment includes that the number of acquisition steps is dependent upon a specified or predicted total number of received medical data packets. The total number of received medical data packets is a total number of medical data packets since the start of the acquisition. The total number may be known before the start of the method and may then be specified accordingly. However, the total number is often unknown before the start of the method since, for example, it may depend upon the medical investigation and/or treatment. However, a value may nevertheless be specified for the total number. An average value, for example, is suitable as a specified value. The average total number may be an average value that was calculated in that numerous medical data packets received in the past have been evaluated with regard to a respective total number of received medical data packets per patient. The average total number may be stored and on execution of the method, may be adopted as a specified total number. Alternatively to this, the total number may be predicted, that is, estimated, for example, dependent upon at least one received additional information item that defines the medical investigation and/or treatment during which the method is carried out. Therefore, no number of acquisition steps is fixedly specified, but rather it depends dynamically upon the total number. This has the consequence that the number of acquisition steps may be adapted to smaller or larger total numbers of medical data packets in order, for example, to achieve a suitable time saving.

Furthermore, one embodiment provides that the number of acquisition steps corresponds to the square root of the specified or predicted total number of received medical data packets. Thus, if, for example, 500 medical data packets are received or expected according to the specified or predicted total number, the number of acquisition steps that are omitted or jumped over by a pointer is, for example, the square root of 500. This depicts how, with little computation effort and therefore quickly, the number of acquisition steps may be dynamically specified.

Another aspect relates to a method for storing medical data. The method includes a continuous acquisition of medical measurement values, for example, of specific absorption rates. The method also includes a continuous creation of medical data packets, each of which includes a start time point, an end time point and a data packet value, wherein the data packet value defines a sum of medical measurement values acquired between the start time point and the end time point. In addition, an execution of at least one of the two computer-implemented methods described above takes place in order to store medical data.

One embodiment of the method provides that, if no medical measurement values are acquired during a time-span, the computer-implemented method is paused. A temporal spacing between the end time point of the last created medical data packet and the start time point of the medical data packet created thereafter corresponds to the time-span. A time specification continues therefore, even if no medical measurement values are currently being acquired. Between the end time point of a first element and the start time point of an element adjoining it, a time-span of greater than 0 may therefore have elapsed. In this way, what is achieved is that no entries into the linked list and/or the data field take place if no medical measurement data at all is acquired and therefore no medical data packets are created. This has the result that less storage space is needed as compared with a procedure in which, without taking account of the time-span, further entries into the linked list and/or the data field are undertaken.

Continuous, in the sense of the invention, means an ever recurring acquisition and/or creation so that, for example, individual measurement values are acquired one after the other and, from these, individual medical data packets are generated. Continuous may also be construed as meaning over and again or repeatedly.

The receiving or obtaining of data or information may, for example, include a receiving or obtaining of the data, for example, by the data processing system from a transmitting entity, or a reading-out of the data from a data store or receiving or obtaining a data stream that contains the data, or the extraction of the data from the data stream, and so forth. For this purpose, wire-bound or wireless data transfer may be used, for example. For example, the data transfer may take place between a hardware interface and/or software interface of the transmitting entity and a hardware interface and/or a software interface of the data processing system.

Further aspects relate to the data processing system, a computer program product and a computer-readable storage medium.

If not otherwise stated, all the steps of the computer-implemented method may be carried out by a data processing system that has at least one data-processing device. For example, the at least one data processing device is configured or adapted to carry out the steps of the computer-implemented method. For this purpose, the at least one data processing device may store, for example, a computer program that contains commands that, when they are executed by the at least one data processing device, cause the at least one data processing device to carry out the computer-implemented method. The computer-implemented method may also be implemented entirely or partially in hardware. The expressions “data processing system” and “at least one data processing device” may be used interchangeably here and in the following. This also applies to corresponding expressions derived therefrom.

In the event that the at least one data processing device contains two or more data processing devices, particular steps performed by the at least one data processing device may also be understood to mean that different data processing devices carry out different steps or different parts of a step. For example, it is not required that each data processing device carries out the steps. In other words, the execution of the steps may be distributed over the two or more data processing devices.

From each embodiment of the respective computer-implemented method, there follows a corresponding embodiment of the method that is not purely computer-implemented in that corresponding steps for generating the medical measurement values are included.

The computer program includes commands. If the commands are carried out by a data processing system, the commands cause the data processing system to carry out the computer-implemented method.

The commands may be present, for example, as program code. The program code may be provided, for example, as binary code or assembler and/or as source code of a programming language, for example, C and/or as a program script, for example, Python.

The computer-readable storage medium is, for example, a physical and/or non-volatile computer-readable storage medium that stores a computer program.

The computer program and the computer-readable storage medium are each computer program products with the commands.

Further features and feature combinations are disclosed in the drawings and their description as well as in the claims. For example, further embodiments must not necessarily include all the features of one of the claims. Further embodiments may have features or combinations of features that are not specified in the claims. Embodiments includes the combination of the individual embodiments.

### BRIEF DESCRIPTION OF THE FIGURES

FIG. 1 depicts a schematic representation of a signal-flow graph of a method for storing medical data according to an embodiment.

FIG. 2 depicts a schematic representation of elements of a linked list according to an embodiment.

FIG. 3 depicts a schematic representation of a magnetic resonance tomography device according to an embodiment.

### DETAILED DESCRIPTION

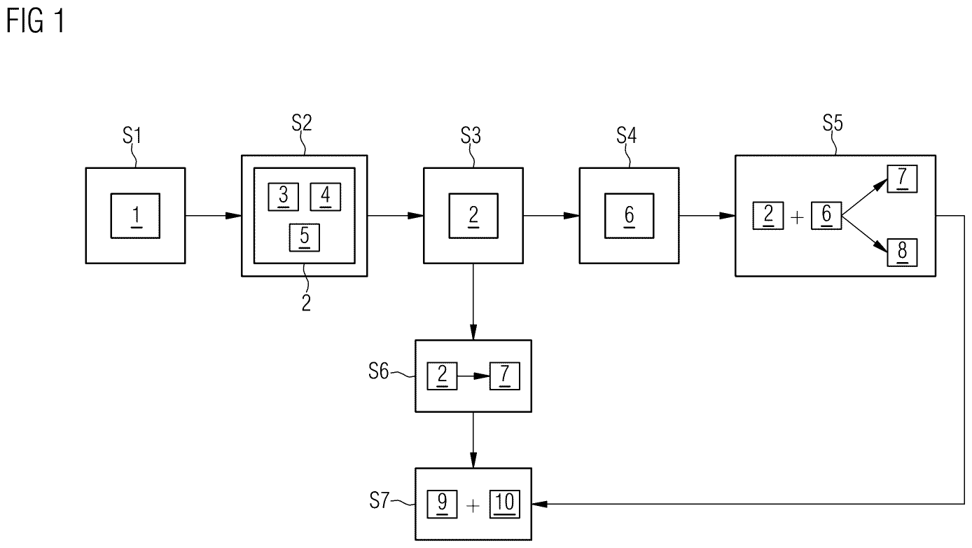

FIG. 1 depicts method steps of a method for storing medical data. In a method step S1, a continuous acquisition of medical measurement values 1 may take place. On the basis of the continuously acquired medical measurement values 1, in a method step S2, a continuous creation of medical data packets 2 may take place. Each medical data packet 2 includes, for example, a start time point 3, an end time point 4 and a data packet value 5 that defines a total of measurement values 1 acquired between the start time point 3 and the end time point 4.

In a method step S3, in one example, a computer-implemented method begins that includes at least one continuous receiving of the medical data packets 2 created one after the other. The data packets 2 created in the method step S2 are thus received, for example, in method step S3 by, for example, a data processing system 23 (see reference sign 23 in FIG. 3) that carries out the computer-implemented method.

In a method step S4, for each received medical data packet 2, a determination of a total value 6 may take place by adding the data packet value 5 according to the respective medical data packet 2 with the data packet values 5 of at least a part of the received medical data packets 2 created temporally before the respective medical data packet 2. The total value 6 is therefore, for example, a current total of the previously acquired medical measurement values 1. In a method step S5, medical data may be stored by storing the respective medical data packet 2 together with the respectively determined total value 6 in a linked list 7 and/or in a data field 8. The linked list 7 and/or the data field 8 are each data structures in order to store data elements in a computer. The computer should be understood herein to be the data processing system 23.

The medical measurement values 1 are, for example, a specific absorption rate that defines a level of energy absorbed per unit time and mass in a body of a patient by way of electromagnetic radiation during a medical investigation and/or treatment, for example, during a magnetic resonance tomography (MRI).

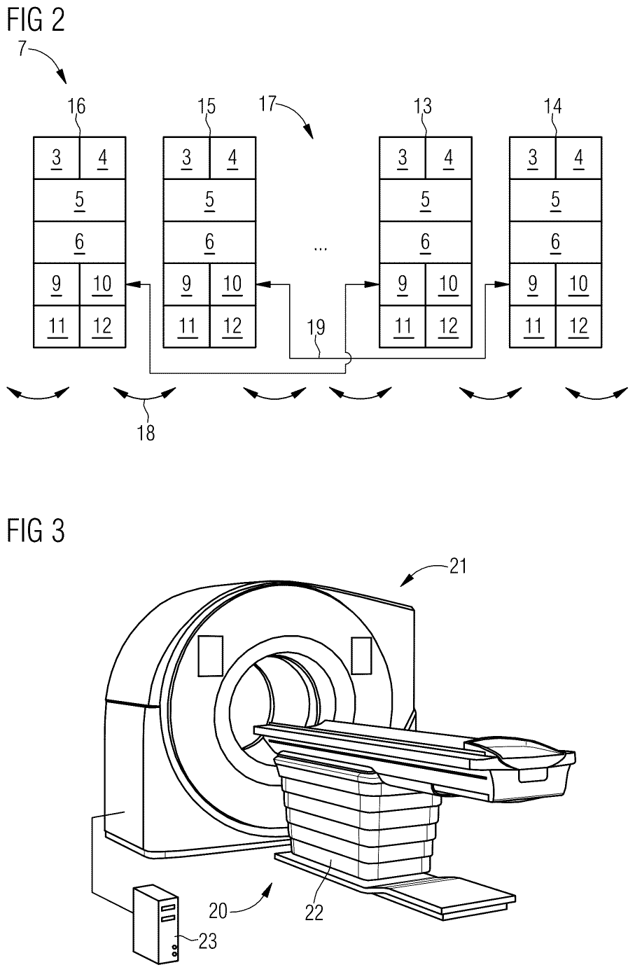

Furthermore, a method step S6 may be provided directly following the method step S3 or after the method step S5. The method step S6 includes storing the medical data by storing each medical data packet 2 in a linked list 7. The respective medical data packet 2 may be understood as a respective element 13, 14, 15, 16 (see reference signs 13, 14, 15, 16 in FIG. 2) of the linked list 7. For each element 13, 14, 15, 16 of the linked list 7, an allocation of at least one first pointer 9 and at least one second pointer 10 takes place. The first pointer 9 is directed to a preceding element 13, 14, 15, 16 that includes the medical data packet 2 that was acquired or created a specified number of acquisition steps before the respective medical data packet 2 currently being considered. The at least one second pointer 10 is directed to a following element 13, 14, 15, 16 that includes the medical data packet 2 that was acquired or created a specified number of acquisition steps after the respective medical data packet 2 currently being considered.

FIG. 2 depicts the linked list 7 and its individual elements 13, 14, 15, 16 in detail. Each element 13, 14, 15, 16 includes, for example, the start time point 3, the end time point 4, the data packet value 5 and the total value 6. In one example, two first pointers 10, 12 and two second pointers 9, 11 may also be provided. The second pointer 11 points here to the directly preceding element 13, 14, 15, 16, for example, in the case of the element 15, to the element 16. The first pointer 12 points here to the directly following element 13, 14, 15, 16, for example, in the case of the element 16, to the element 15. To elucidate these pointers 11, 12, first arrows 18 are sketched in, each being directed to immediately adjacent elements 13, 14, 15, 16.

The second pointer 9 may point to the preceding element 13, 14, 15, 16 that is situated a specified number of acquisition steps before the element 13, 14, 15, 16. In the case of the element 13, this is, for example, the element 15. The first pointer 10 may point to the following element 13, 14, 15, 16 that is situated a specified number of acquisition steps after the element 13, 14, 15, 16. In the case of the element 15, this is, for example, the element 13. To elucidate these pointers 9, 10, second arrows 19 are sketched in, each being directed to the elements 13, 14, 15, 16 removed by the specified number of acquisition steps.

Between the element 16 and the element 13, but also between the element 15 and the element 14, there is a plurality of other elements 13, 14, 15, 16, wherein the number of other elements 13, 14, 15, 16 corresponds to the number of acquisition steps. The plurality of other elements 13, 14, 15, 16 are here indicated with three dots in a gap between the element 15 and the element 13 into which reference sign 17 points.

Numerous elements 13, 14, 15, 16 from the linked list 7 may be included. The four outlined elements 13, 14, 15, 16 are to be regarded as purely exemplary. The linked list 7 may include a first element 13, 14, 15, 16, that is, a start element, and a last element 13, 14, 15, 16, that is, an end element (not shown here), wherein all the other elements 13, 14, 15, 16 of the linked list 7 are situated between the first element 13, 14, 15, 16 and the last element 13, 14, 15, 16.

The number of acquisition steps may be specified or may be determined dependent upon a specified or predicted total number of received medical data packets 2. For example, it results from the square root of the specified or predicted total number.

If the computer-implemented method is, for example, paused because temporarily no medical measurement values 1 are acquired, the computer-implemented method may be paused, wherein a temporal spacing between the end time point 4 of the medical data packet 2 created immediately beforehand from the start time point 3 of the medical data packet 2 created immediately thereafter corresponds to the time-span that is paused. Alternatively, zero elements may be stored, that is, elements 13, 14, 15, 16 that have only the start time point 3 and the end time point 4, but are otherwise empty, that is, that have, for example, no data packet value 5 and the same total value 6 as the preceding element 13, 14, 15, 16. In the case of a zero element, therefore, the total value 6 of the preceding element 13, 14, 15, 16 is therefore added to the number 0.

It may be provided that at least one time interval is specified, wherein on determination of the total value 6 for the respective medical data packet 2, only the received medical data packets 2 whose start time point 3 and/or end time point 4 are in the same specified time interval as the respective medical data packet 2 are taken into account. The specified time interval may be 10 seconds and/or 6 minutes long.

FIG. 3 depicts schematically an exemplary implementation of a magnetic resonance imaging system. A magnetic resonance tomography (MRI) device 20 is shown. The MRI device 20 includes, for example, a housing that defines an opening and a main magnet arrangement 21 that is configured so that it creates a main magnetic field, also designated a polarization magnetic field, within the opening. The MRT device 20 includes, for example, an RF (radio frequency) system that is configured so that it applies RF pulses to a target material, for example, a body part of a patient who is lying on a support 22 that is situated in the opening and receives magnetic resonance signals that are emitted by the target material. For example, the main magnet arrangement 21 may create a homogeneous main magnetic field B0 as the main magnetic field and at least one high frequency coil of a high frequency system may emit an excitation field B20. The MRI device 20 includes a data processing system 23 with at least one computing facility.

According to MR techniques, the target material is exposed to the main magnetic field so that the nuclear spins in the target material precess about the direction of the main magnetic field with their characteristic Larmor frequency. A net magnetic moment MZ is created in the direction z of the main magnetic field and the randomly oriented magnetic moments of the nuclear spins cancel each other out in the X-Y plane.

If the target material is then subjected to the transmission radio frequency magnetic field that exists, for example, in the X-Y plane and is close to the Larmor frequency, the net magnetic moment rotates out of the Z-direction and creates a net magnetic moment in the plane the projection of which in the X-Y plane rotates at the Larmor frequency. As a reaction thereto, MR signals are emitted by the excited spins when they return to their state before the excitation. The emitted MR signals are acquired, for example, by at least one high frequency coil and/or one or more of dedicated detection coils, digitized in a receiving channel of a high frequency controller of the high frequency system and processed by at least one processor of the at least one computing apparatus in order, for example, to reconstruct an MR image using a known reconstruction technique.

For example, gradient coils of the MRI device may create magnetic field gradients GX, GY and GZ for position encoding of the MR signals. Accordingly, MR signals are only emitted by nuclei of the target material that correspond to the respective Larmor frequency. For example, GZ is used together with a bandwidth-limited RF pulse in order to select a slice perpendicular to the Z-direction and may consequently also be designated the slice selection gradient. Alternatively, GX, GY and GZ may be used in any desired pre-defined combination with a bandwidth-limited high frequency pulse in order to select a slice perpendicular to the vector sum of the gradient combination. The gradient coils may be supplied with current by the respective amplifiers in order to create the respective gradient fields in the X, Y and Z directions. Each amplifier may include a corresponding digital-to-analog converter that is controlled by a sequence controller in order to create gradient pulses at pre-defined time points.

It should be noted that the components of the MRI device 20 may also be differently arranged than in the arrangement shown in FIG. 3 or as described. For example, the gradient coils may be arranged within the opening, similarly to the at least one high frequency coil.

The sequence control unit of the at least one computing apparatus may control the creation of high frequency pulses by an emitter channel of the high frequency control unit and a high frequency power amplifier of the high frequency system.

The at least one computing apparatus may receive the real and imaginary parts from analogue-to-digital converters of the receive channel and based thereon, may reconstruct the MR image according to a known method.

Altogether, the examples show an output optimization for value sums in doubly linked lists 7. Conventionally, the integral values (data packet values 5) of the individual time portions with their start and end time points (start time point 3 and end time point 4) have been stored in doubly linked lists 7. For average value formation, it was necessary to iterate from the start or the end of the list 7 across all the entries and to total the contents in the time region of interest and to divide them by the interval lengths in order to form the average value. Therein, time regions the integral of which is 0 do not have to be entered into the list 7 (pauses) and the time portions themselves may be of variable length (saves repetition of list elements).

The optimization consists of two parts:
a) in addition to the individual integral values (data packet values 5), on creation of the list 7, the total integral values (total values 6) since the start of the list 7 are also entered into the respective list elements (elements 13, 14, 15, 16); and/or
b) in addition to the pointers 11, 12 to the preceding item or following item, pointers 9, 10 to the x-th preceding item or following item are also created when the list 7 is established.

With these new entries, in the first place, the repeated addition of integral values is spared. Secondly, it is possible to navigate substantially faster in the list 7 with the x-pointers 11, 12.

Rather than linked lists 7, arrays (data fields 8) may also be used. These have the advantage that the pointers 9, 10, 11, 12 may be spared and list elements (elements 13, 14, 15, 16) may be accessed faster, although they also have the following disadvantages:
a) arrays cannot be changed in size. This requires reallocation and copying. In the real-time context, a storage allocation is also critical and, for linked lists 7, at program start, a sufficiently large pool may be allocated, that then does not need to be reallocated.
b) with arrays, elements 13, 14, 15, 16 cannot be deleted at the start, or only by copying all the elements 13, 14, 15, 16 across. A ring buffer is also only usable for a fixed array length. For moving average values over fixed time regions (such as required, for example, in SAR monitoring), the deletion of aged time regions is a standard requirement.
c) if not only one list 7, but a plurality thereof in different lengths are needed, in the case of arrays, a maximum number must always be allocated, that requires significantly more memory storage than a total pool of all the elements 13, 14, 15, 16 for linked lists 7. For SAR monitoring, for example, for the prediction, significantly longer time regions are relevant than for an online monitoring (real time monitoring).

The number of elements 13, 14, 15, 16 in linked lists 7 that cover the time regions from 10 minutes to 60 minutes with a resolution of, for example, 0.5 seconds, depending upon the length of the pause times, is in the order of 1000 to 5000. According to the previous solution, for the average value formation over, for example, 6 minutes, approximately 700 elements 13, 14, 15, 16 must be added and, dependent upon the length of the time region, iteration must take place over up to N/2 (N being the length of the list 7) elements 13, 14, 15, 16. According to the invention, it is possible to navigate to the start and the end element 13, 14, 15, 16, that is reachable significantly faster with the X-pointers 9, 10 and the difference of the total values may be calculated. Performance increases by a factor of 1.8 to 5.3 may be made possible.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A computer-implemented method for storing medical data, the method comprising:
continuous receiving sequentially created medical data packets, each of which comprises a start time point, an end time point, and a data packet value that defines a total of medical measurement values acquired between the start time point and the end time point;
for each received medical data packet, determining a total value by adding the data packet value of a respective medical data packet with data packet values of one or more received medical data packets created temporally before the respective medical data packet; and
storing the medical data by storing the respective medical data packet together with the determined total value in a linked list and/or in a data field.

2. The computer-implemented method of claim 1, wherein the medical measurement values specify a specific absorption rate which defines a level of energy absorbed per unit time and mass in a body of a patient by way of electromagnetic radiation during a medical investigation and/or treatment.

3. The computer-implemented method of claim 1, wherein at least one time interval is specified and, on determination of the total value for the respective medical data packet, only received medical data packets of the sequentially created medical data packets whose start time point and/or end time point are in the same specified time interval as the respective medical data packet are taken into account.

4. The computer-implemented method of claim 3, wherein the at least one specified time interval is at least 10 seconds long.

5. The computer-implemented method of claim 1, wherein the medical data is stored in the linked list, wherein, together with the established total value, each medical data packet of the sequentially created medical data packets forms a respective element of the linked list, wherein at least one first pointer to a following element and at least one second pointer to a preceding element is associated with the respective element.

6. The computer-implemented method of claim 5, wherein the following element comprises a medical data packet that was acquired directly after the respective medical data packet, and the preceding element comprises a medical data packet that was acquired directly before the respective medical data packet.

7. The computer-implemented method of claim 5, wherein the following element comprises a medical data packet that was acquired a specified number of acquisition steps after the respective medical data packet, and the preceding element comprises a medical data packet that was acquired the specified number of acquisition steps before the respective medical data packet.

8. A computer-implemented method for storing medical data, the method comprising:
continuous receiving of sequentially created medical data packets, each of which comprises a start time point, an end time point, and a data packet value that defines a total of medical measurement values acquired between the start time point and the end time point;
storing medical data by storing the sequentially created medical data packets in a linked list, wherein a respective medical data packet of the sequentially created medical data packets forms a respective element of the linked list; and
for the respective element of the linked list, allocating at least one first pointer to a following element and at least one second pointer to a preceding element, wherein the following element comprises a medical data packet that was created a specified number of acquisition steps after the respective medical data packet, and the preceding element comprises the medical data packet that was created the specified number of acquisition steps before the respective medical data packet.

9. The computer-implemented method of claim 8, wherein the specified number of acquisition steps is dependent upon a specified or predicted total number of received medical data packets.

10. The computer-implemented method of claim 9, wherein the specified number of acquisition steps corresponds to a square root of the specified or predicted total number.

11. A method for storing medical data, comprising:
continuously acquiring medical measurement values;
continuously creating medical data packets, each of which comprises a start time point, an end time point and a data packet value that defines a total of medical measurement values acquired between the start time point and the end time point; and
for each created medical data packet, determining a total value by adding the data packet value of a respective medical data packet with data packet values of one or more received medical data packets created temporally before the respective medical data packet; and
storing the medical data by storing the respective medical data packet together with the determined total value in a linked list and/or in a data field.

12. The method of claim 11, wherein if for a time-span, no medical measurement values are acquired, the method is paused, wherein a temporal spacing between the end time point of the last created medical data packet and the start time point of a medical data packet created thereafter corresponds to the time-span.
